(19) United States
(12) Patent Application Publication
(10) Pub. No.: US 2022/0383160 A1
(43) Pub. Date: Dec. 1, 2022

(54) SAMPLE ANALYSIS DEVICE, SAMPLE ANALYSIS METHOD, PHARMACEUTICAL ANALYSIS DEVICE AND PHARMACEUTICAL ANALYSIS METHOD

(71) Applicant: SHIMADZU CORPORATION, Kyoto-shi (JP)

(72) Inventor: Yusuke NAKAI, Kyoto-shi (JP)

(21) Appl. No.: 17/827,896

(22) Filed: May 30, 2022

(30) Foreign Application Priority Data
Jun. 1, 2021 (JP) 2021-092141

(51) Int. Cl. G06N 5/04; G06N 7/00; G16C 20/50

(52) U.S. Cl. CPC G06N 5/04; G06N 7/005; G16C 20/50

(57) ABSTRACT

A sample analysis device includes an acquirer that acquires quantitative information of a test substance present in a sample, an estimator that reads a generalized reaction model obtained by generalization of a plurality of reaction models from a storage device and estimates a posterior distribution of a parameter of the generalized reaction model using Bayesian inference, and a calculator that calculates a confidence interval or a quantile of the quantitative information of a test substance in any period of time or calculates a confidence interval of a quantile in a period of time until the quantitative information of a test substance reaches a predetermined specification limit, based on the posterior distribution of a parameter estimated by the estimator.

### BACKGROUND

### Technical Field

The present invention relates to a sample analysis device and a sample analysis method for analyzing a test substance included in a sample, and a pharmaceutical analysis device and a pharmaceutical analysis method for analyzing an active ingredient or the like contained in a formulation or the like.

### Description of Related Art

A stability test is carried out to assess a change in a pharmaceutical over time. In this test, a period (shelf life) in which it can be ensured that the value of an active ingredient of a pharmaceutical is in a reference range or that the value of impurities are equal to or smaller than a reference value is calculated. Generally, an active ingredient and impurities are identified and quantified by liquid chromatography in regard to a pharmaceutical stored for a certain period of time in a thermo-humidistat chamber or the like, and the shelf life is calculated based on the results.

It is necessary to store a pharmaceutical for a long period of time to carry out the stability test. In order to shorten this period, a method of predicting a shelf life by using a reaction model function (extrapolation along a time axis) or a method of predicting a shelf life under a low temperature (low humidity) condition based on a decomposition amount under a high temperature (high humidity) condition using the Arrhenius equation (extrapolation along a temperature axis) is performed. Extrapolation along a time axis is disclosed in “Assessment of stability test data for pharmaceutical products containing a new active ingredient” published by Japan Pharmaceutical Manufacturers Association and the Pharmaceutical Publishing Center in March, 2005, for example. Extrapolation along a humidity axis is disclosed in the “WEB page for ASAPrime (a software package based on the Accelerated Stability Assessment Program),” by FreeThink Technologies, Inc., [Searched on May 24, 2021], <URL: https://www.ms-scientific.com/products/lifescience/asapprime>, for example.

### SUMMARY

A result of a pharmaceutical stability test is required to not only provide prediction in regard to whether a model fits well but also setting of a reasonable distribution and a confidence interval. Conventionally, in a method using a model function, an approach of setting a wider confidence interval in consideration of a model error or acquiring data of a sufficiently long period is taken. However, due to such an approach, there is a problem that a shelf life is excessively short or a problem that it takes a long period of time to acquire necessary data.

An object of the present invention is to provide a sample analysis device, a sample analysis method, a pharmaceutical analysis device and a pharmaceutical analysis method that enable presentation of a reasonable confidence interval.

A sample analysis device according to one aspect of the present invention includes an acquirer that acquires quantitative information of a test substance present in a sample, an estimator that reads a generalized reaction model obtained by generalization of a plurality of reaction models from a storage device and estimates a posterior distribution of a parameter of the generalized reaction model using Bayesian inference, and a calculator that calculates a confidence interval or a quantile of the quantitative information of a test substance in any period of time or calculates a confidence interval of a quantile in a period of time until the quantitative information of a test substance reaches a predetermined specification limit, based on the posterior distribution of a parameter estimated by the estimator.

A sample analysis device according to another aspect of the present invention includes an acquirer that acquires quantitative information of a test substance present in a sample, an estimator that reads a reaction model stored in a storage device and estimates a posterior distribution of a parameter using Bayesian inference by combining an Arrhenius equation or a modified Arrhenius equation with the reaction model, and a calculator that calculates a confidence interval or a quantile of the quantitative information of a test substance in any period of time or calculates a confidence interval of a quantile in a period of time until the quantitative information of a test substance reaches a predetermined specification limit, based on the posterior distribution of a parameter estimated by the estimator.

The present invention is also directed to a sample analysis method, a pharmaceutical analysis device and a pharmaceutical analysis method.

Other features, elements, characteristics, and advantages of the present disclosure will become more apparent from the following description of preferred embodiments of the present disclosure with reference to the attached drawings.

### BRIEF DESCRIPTION OF THE DRAWING

FIG. 1 is a diagram showing the configuration of a sample analysis device according to the present embodiment;

FIG. 2 is a block diagram showing the functions of the sample analysis device according to the present embodiment;

FIG. 3 is a diagram showing extrapolation along a time axis;

FIG. 4 is a diagram showing an example of a reaction model;

FIG. 5 is a diagram showing extrapolation along a temperature axis;

FIG. 6 is a flowchart showing an analysis method according to a first embodiment;

FIG. 7 is a diagram showing simulation data;

FIG. 8 is a diagram showing a posterior distribution of a peak area ratio estimated with use of the Bayesian inference and the simulation data;

FIG. 9 is a flowchart showing an analysis method according to a modified example 1 of the first embodiment;

FIG. 10 is a flowchart showing an analysis method according to a second embodiment; and

FIG. 11 is a flowchart showing an analysis method according to a modified example of the second embodiment.

### DETAILED DESCRIPTION

A sample analysis device, a sample analysis method, a pharmaceutical analysis device and a pharmaceutical analysis method according to embodiments of the present invention will now be described with reference to the attached drawings.

### (1) Configuration of Sample Analysis Device

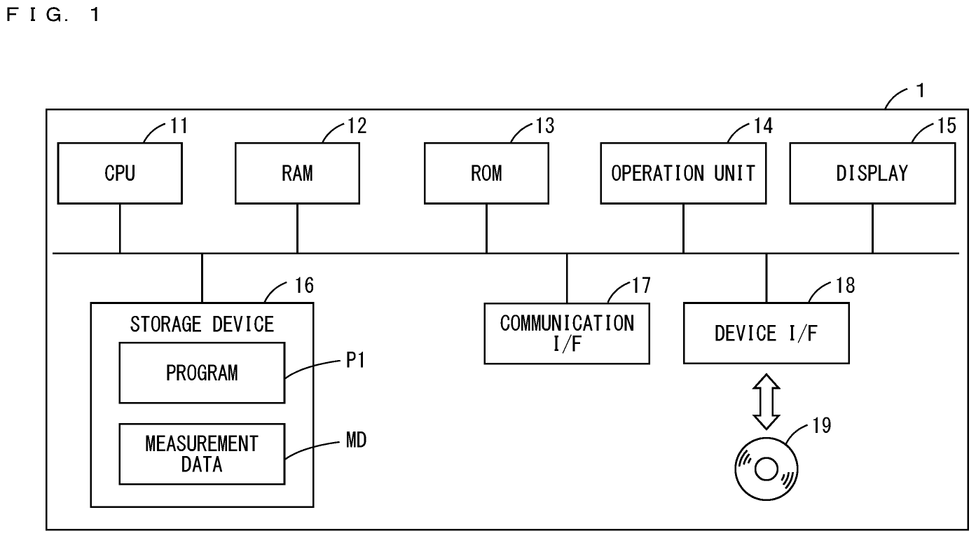

FIG. 1 is a diagram showing the configuration of the sample analysis device 1 according to embodiments. The sample analysis device 1 of the present embodiment acquires measurement data MD of a sample obtained in a liquid chromatograph, a gas chromatograph, a mass spectrometer or the like. The measurement data MD has quantitative information of a test substance present in a sample. Specifically, the measurement data MD includes data in regard to a peak area ratio of the test substance present in the sample. In the present embodiment, a pharmaceutical (a formulation or a drug substance) is used as a sample, by way of example. Specifically, in the present embodiment, the measurement data MD includes data in regard to the ratio of a peak area of impurities with respect to a peak area of an active ingredient included in a pharmaceutical. The measurement data MD has data about a peak area ratio in regard to a plurality of points in time.

The sample analysis device 1 of the present embodiment is constituted by a personal computer. As shown in FIG. 1, the sample analysis device 1 includes a CPU (Central Processing Unit) 11, a RAM (Random Access Memory) 12, a ROM (Read Only Memory) 13, an operation unit 14, a display 15, a storage device 16, a communication interface (I/F) 17 and a device interface (I/F) 18.

The CPU 11 controls the sample analysis device 1 as a whole. The RAM 12 is used as a work area for execution of a program by the CPU 11. Various data, a program and the like are stored in the ROM 13. The operation unit 14 receives an input operation performed by a user. The operation unit 14 includes a keyboard, a mouse, etc. The display 15 displays information such as a result of analysis. The storage device 16 is a storage medium such as a hard disc. A program P1 and the measurement data MD are stored in the storage device 16.

The program P1 estimates a predictive value (a posterior distribution) of a parameter of a generalized reaction model obtained by generalization of a plurality of reaction models with use of the Bayesian inference. Further, the program P1 estimates a predictive value of a parameter (a posterior distribution) using the Bayesian inference by combining the Arrhenius equation or a modified Arrhenius equation, and a reaction model. Further, the program P1 calculates a confidence interval or a quantile of quantitative information of a test substance in any point in time based on the estimated posterior distribution of a parameter. Further, the program P1 calculates a confidence interval or a quantile in a period of time until the quantitative information of a test substance reaches a predetermined specification limit based on the estimated posterior distribution of a parameter.

The communication interface 17 is an interface that communicates with another computer through wireless or wired communication. The device interface 18 is an interface that accesses a storage medium 19 such as a CD, a DVD or a semiconductor memory.

### (2) Functional Configuration of Sample Analysis Device

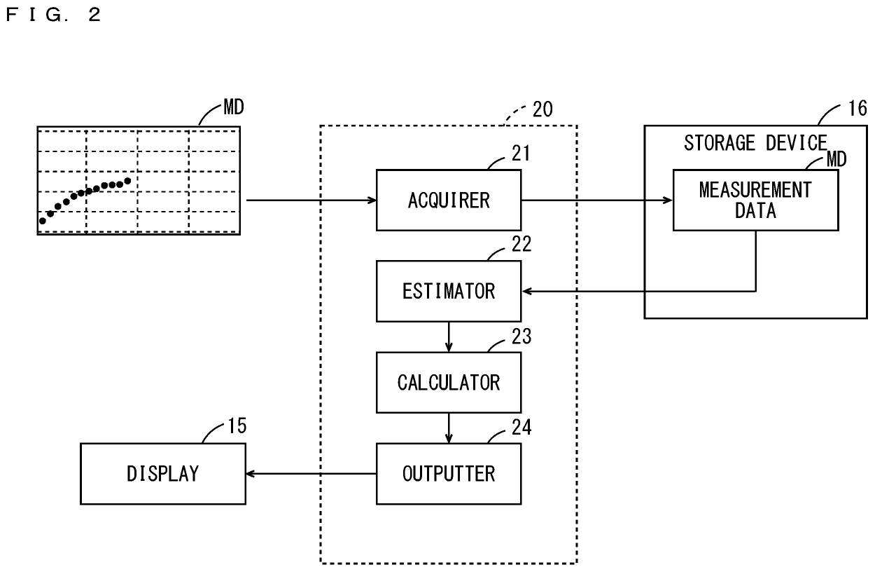

FIG. 2 is a block diagram showing the functional configuration of the sample analysis device 1. In FIG. 2, a controller 20 is a function that is implemented by execution of the program P1 by the CPU 11 while the CPU 11 uses the RAM 12 as a work area. The controller 20 includes an acquirer 21, an estimator 22, a calculator 23 and an outputter 24. That is, the acquirer 21, the estimator 22, the calculator 23 and the outputter 24 are the functions implemented by execution of the program P1. In other words, each of the functions 21 to 24 is a function included in the CPU 11.

The acquirer 21 receives the measurement data MD. The acquirer 21 receives the measurement data MD from another computer, an analysis device and the like via the communication interface 17, for example. Alternatively, the acquirer 21 receives the measurement data MD saved in the storage medium 19 via the device interface 18.

The estimator 22 estimates a posterior distribution of a parameter of a generalized reaction model using the Bayesian inference and the measurement data MD. The generalized reaction model is a model obtained by generalization of a plurality of reaction models. Further, the estimator 22 estimates a posterior distribution of a parameter with use of the Bayesian inference by combining the Arrhenius equation or a modified Arrhenius equation, and a reaction model.

The calculator 23 calculates a confidence interval or a quantile of quantitative information of a test substance at any point in time based on the posterior distribution of a parameter estimated by the estimator 22. Further, the calculator 23 calculates a confidence interval or a quantile in a period of time until quantitative information of a test substance reaches a predetermined specification limit based on the posterior distribution of a parameter estimated by the estimator 22.

The outputter 24 displays a confidence interval or a quantile of quantitative information of a test substance in the display 15. The outputter 24 also displays a confidence interval or a quantile in a period of time until quantitative information of a test substance reaches the predetermined specification limit.

The program P1 is saved in the storage device 16, by way of example. In another embodiment, the program P1 may be saved in the storage medium 19 for provision. The CPU 11 may access the storage medium 19 via the device interface 18 and may save the program P1 saved in the storage medium 19 in the storage device 16 or the ROM 13. Alternatively, the CPU 11 may access the storage medium 19 via the device interface 18 and may execute the program P1 saved in the storage medium 19.

### (3) Prediction Based on Measurement Data

(3-1) Extrapolation Along Time Axis

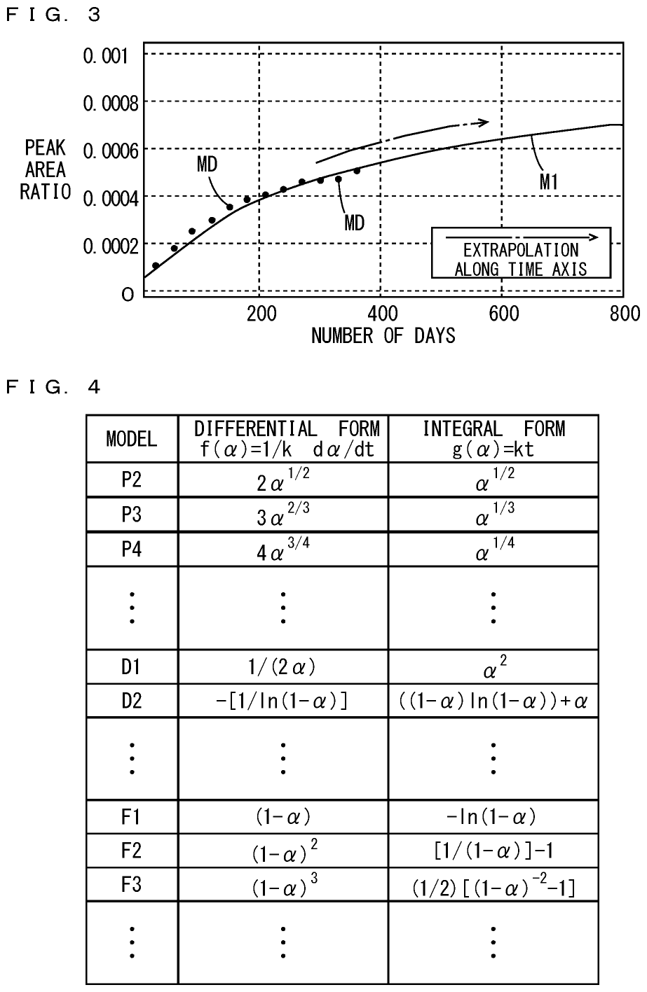

Extrapolation along a time axis based on the measurement data MD which is the basis for performing the analysis method of the present embodiment will be described before description of an analysis method performed by the sample analysis device 1 of the present embodiment. FIG. 3 is a diagram showing extrapolation along the time axis. In FIG. 3, the abscissa indicates the number of days (time), and the ordinate indicates the ratio of a peak area of impurities with respect to a peak area of a main component. In case of a pharmaceutical, the ordinate indicates the ratio of a peak area of impurities with respect to a peak area of an active ingredient.

In FIG. 3, the plotted points indicate the measurement data MD. The measurement data MD is the data of peak area ratios acquired on a plurality of days. In the example of FIG. 3, the measurement data MD is the data acquired from the first day to about the 400th day. Regression is performed on this acquired measurement data MD, so that the model M1 shown in the diagram is fitted. The model M1 is fitted, so that the peak area ratios on the future days such as the 600th day or the 800th day are estimated based on the measurement data MD of up to about the 400th day. The model M1 is fitted in this manner, so that the peak area ratio is extrapolated along the time axis. Similarly, it is possible to interpolate the peak area ratio along the time axis by fitting the model M1.

FIG. 4 is a diagram showing an example of a reaction model. In FIG. 4, each reaction model is represented in two forms: a differential form and an integral form. In the diagram, a represents a conversion rate, which is a value from 0 to 1 indicating the progress of reaction. k represents a reaction rate constant. It is possible to perform extrapolation (and interpolation) along the time axis by applying any reaction model to the measurement data MD and estimating a parameter such as k by regression. In a case in which regression is performed with use of a differential form, it is necessary to modify a differential form into da/dt=kf(α) to solve a differential equation. However, a differential form is characterized that it is easier to generalize a model formula with use of a differential than with an integral form.

(3-2) Extrapolation Along Temperature Axis

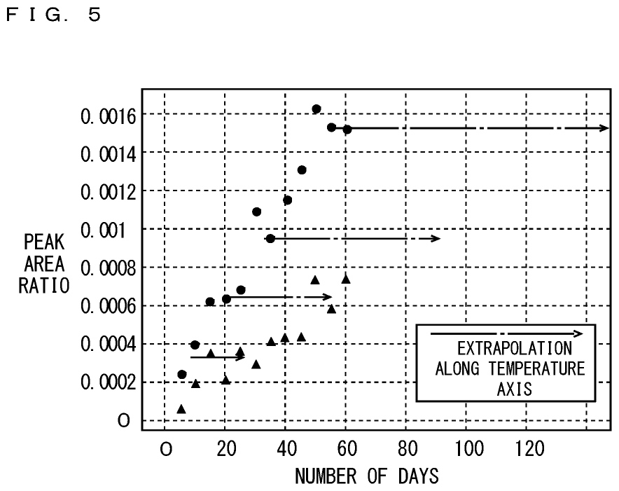

Subsequently, extrapolation along a temperature axis based on the measurement data MD which is the basis for performing the analysis method of the present embodiment will be described. FIG. 5 is a diagram showing the extrapolation along the temperature axis. In FIG. 5, the abscissa indicates the number of days (time), and the ordinate indicates the ratio of a peak area of impurities with respect to a peak area of a main component. In case of a pharmaceutical, the ordinate indicates the ratio of a peak area of impurities with respect to a peak area of an active ingredient.

Similarly to FIG. 3, the plotted points are also the measurement data MD and the data of peak area ratios acquired on a plurality of days in FIG. 5. In FIG. 5, the black dots indicate the measurement data MD acquired under high temperature conditions (severe condition), and the black triangles indicate the measurement data MD acquired under low temperature conditions (normal storage conditions). In the example of FIG. 5, the measurement data MD acquired under either high temperature conditions or low temperature conditions is the data acquired from the first day to about the 60th day. Further, it is possible to predict the data to be acquired under low temperature conditions from the measurement data MD acquired under high temperature conditions by using the following Arrhenius equation. Thus, the peak area ratios to be acquired on the future days such as the 100th day, the 200th day, after 1 year and after 2 years under low temperature conditions (normal storage condition) are estimated. In this manner, the peak area ratios are extrapolated along the temperature axis.

A reaction rate constant k with respect to single reaction does not change in a case in which the temperature and humidity are constant. However, in a case where the temperature and humidity change, the reaction rate constant k can be considered to be represented by the Arrhenius equation expressed by the formula 1 or the modified Arrhenius equation expressed by the formula 2.

$$[ Formula 1 ]$$

$$k = A exp ( - \frac{E}{R T} ) \qquad ( 1 )$$

$$[ Formula 2 ]$$

$$k = A exp ( - \frac{E}{R T} + B ⨯ H ) \qquad ( 2 )$$

In the formula 1 and the formula 2, R represents a gas constant, T represents an absolute temperature and H represents a relative humidity. Further, A represents a frequency factor, E represents activation energy and B represents a parameter in regard to humidity. While being unique to each reaction, the parameters (A, E and B) are not necessarily unique under conditions in which physical properties change such as a case of non-crystallization or a case of extremely high temperature and high humidity. In a case where a period of time required until a sample reaches a certain decomposition amount is represented by t, “k×t” is constant. Therefore, it is possible to utilize the results of measurement under severe conditions to predict data to be acquired under normal storage conditions by obtaining the parameters (A, E and B) in regard to unknown reaction based on the measurement data MD acquired at a plurality of temperatures or humidities. Although the extrapolation along the temperature axis is described here, the similar method is also performed in regard to extrapolation along a humidity axis.

### (4) First Embodiment

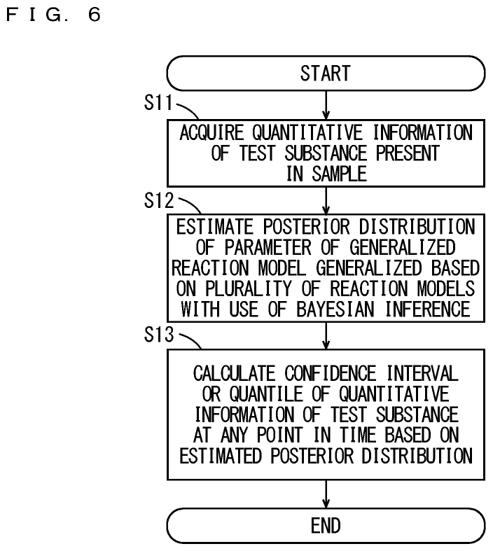

Next, an analysis method according to a first embodiment will be described with reference to the flow chart of FIG. 6. The analysis method of the first embodiment is the extrapolation along the time axis with use of a reaction model described in (3-1). The flowchart of FIG. 6 shows a process to be executed by the CPU 11 shown in FIG. 1. That is, the flowchart of FIG. 6 describes a process to be executed by each of the functions 21 to 24 shown in FIG. 2 when the CPU 11 runs the program P1 while utilizing the hardware resources such as the RAM 12.

In the step S11, the acquirer 21 acquires quantitative information of a test substance present in a sample. Specifically, the acquirer 21 acquires data in regard to the ratio of a peak area of impurities with respect to a peak area of an active ingredient included in a pharmaceutical. Next, in the step S12, the estimator 22 estimates a posterior distribution of a parameter of a generalized reaction model obtained by generalization of a plurality of reaction models with use of the Bayesian inference. The generalized reaction model is stored in the storage device 16.

The generalized reaction model will be described. When it is difficult to select one particular reaction model from the measurement data MD, a plurality of reaction models are generalized. The formula 3 and the formula 4 are examples of a generalized reaction model.

$$[ Formula 3 ]$$

$$\frac{d α}{d t} = {k}_{1} \cdot 2 {α}_{1}^{\frac{1}{2}} + {k}_{2} \cdot \frac{1}{2 {α}_{2}} \qquad ( 3 )$$

$$[ Formula 4 ]$$

$$\frac{d α}{d t} = {k ( 1 - α )}^{n} \qquad ( 4 )$$

The formula 3 is an example for constructing a generalized reaction model by summation of a plurality of reaction models. This generalized reaction model is a model obtained when a P2 model and a D1 model out of the reaction models shown in FIG. 4 are summed. The formula 4 is an example for constructing a generalized reaction model by inclusion of a plurality of reaction models. This generalized reaction model is a model that includes an F1 model and an F2 model out of the reaction models shown in FIG. 4. Such a plurality of generalized reaction models are stored in the storage device 16.

The estimator 22 uses the generalized reaction models expressed by the formula 3, the formula 4 and the like and applies the measurement data MD acquired by the acquirer 21 to the generalized reaction models to perform the Bayesian inference, and acquires a posterior distribution of a parameter.

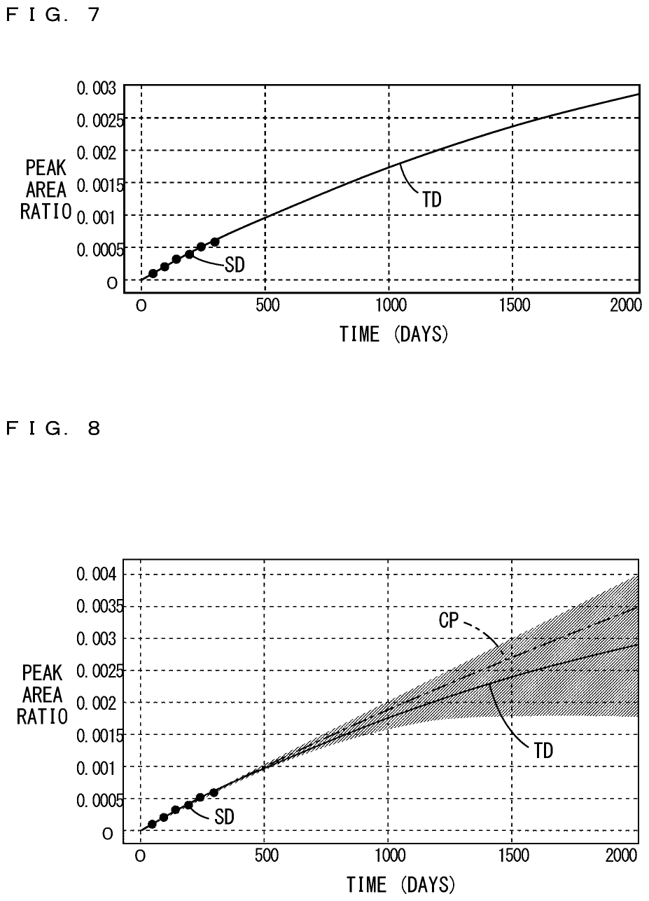

An inventive example of the analysis method according to the present embodiment will be described with use of simulation data SD instead of the measurement data MD. FIG. 7 is a diagram showing the simulation data SD. The simulation data SD is data of peak area ratios from 0 to 300th days. This simulation data SD is created based on a reaction function TD (true function).

FIG. 8 shows a posterior distribution of a reaction model estimated with use of the Bayesian inference after a generalized reaction model is applied to the simulation data SD shown in FIG. 7. In FIG. 8, the hatched region indicates the 95% confidence interval of the posterior distribution of a reaction model. Further, the one-dot and dash line of FIG. 8 indicates the median of the posterior distribution of the reaction model. Further, the solid line in FIG. 8 indicates the reaction function TD (true function) of the simulation data SD shown in FIG. 7.

In order to obtain a probability distribution shown in FIG. 8, an appropriate prior distribution is provided to the generalized reaction model, and the Bayesian inference is performed with use of the simulation data SD. After a warm-up period of a predetermined step has elapsed, the Bayesian inference is performed by execution of the Markov chain Monte Carlo method (MCMC method) of a predetermined step for statistic calculation.

Returning to the flow chart of FIG. 6. Next, in the step S13, the calculator 23 calculates a confidence interval or a quantile of quantitative information of a test substance at any point in time based on the posterior distribution of the reaction model estimated by the estimator 22. That is, the calculator 23 calculates a confidence interval or a quantile of a value of peak area ratio at any point in time. For example, the calculator 23 calculates a confidence interval or a quantile at a point in time such as one year later, two years later or three years later in regard to a peak area ratio of impurities with respect to an active ingredient included in a pharmaceutical. The calculated confidence interval or the calculated quantile may be displayed in the display 15 by the outputter 24.

Even in a case in which prediction is made by a combination of a plurality of reaction models, when a value of a parameter with which an error with respect to acquired data is reduced is calculated, the effect of the analysis method of the present embodiment is not obtained. The method of calculating the value of a parameter with which an error is reduced is excellent in obtaining a model function that fits well from an expressive model. However, the method is not appropriate in a case in in which a confidence interval is desirably calculated in consideration of a possibility of representing another model.

For example, consider a case in which a reaction model y=ax+bx\hat{ }2 is constructed as a model obtained by summation of a linear model y=ax and a quadratic function model y=bx\hat{ }2. This model can express the two models and the model obtained by summation of the two models. Even in a case in which the acquired data “actually represents a linear model but is a quadratic function due to a measurement error,” when fitting is performed with use of this model so as to reduce an error, a quadratic function is obtained, and the possibility of being a linear model is not considered.

In the present embodiment, an approach based on the Bayesian theory is applied to this model to estimate a parameter. In the Bayesian inference, a value of a parameter with which an error is reduced is not obtained but a “distribution” of a parameter is acquired after setting a reasonable error distribution or a prior distribution. Therefore, even in the above-mentioned case of “actually representing a linear model but being a quadratic function due to a measurement error,” it is possible to obtain a distribution of a parameter in consideration of a case in which a linear model is seen due to an error. In the example of generalization by summation expressed by the formula 3, a case where reactions occur in parallel can also be considered. That is, a model can be constructed even in a case in which two reactions of the same type are occurring in parallel. On the other hand, in the example of generalization by inclusion expressed by the formula 4, there is an advantage that the number of parameters can be reduced as compared to the method using summation.

### (5) Modified Example 1 of First Embodiment

In the above-mentioned first embodiment, the calculator 23 calculates a confidence interval or a quantile of quantitative information of a test substance at any point in time based on the posterior distribution of a parameter estimated by the estimator 22. In the modified example 1 of the first embodiment, a confidence interval or the like in a period of time until quantitative information of a test substance reaches a predetermined specification limit is calculated.

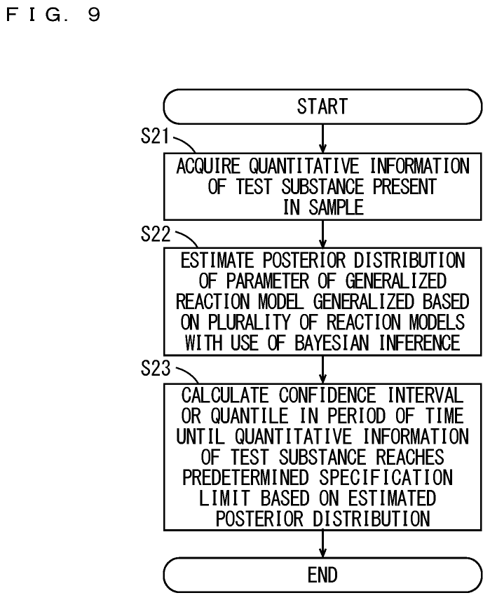

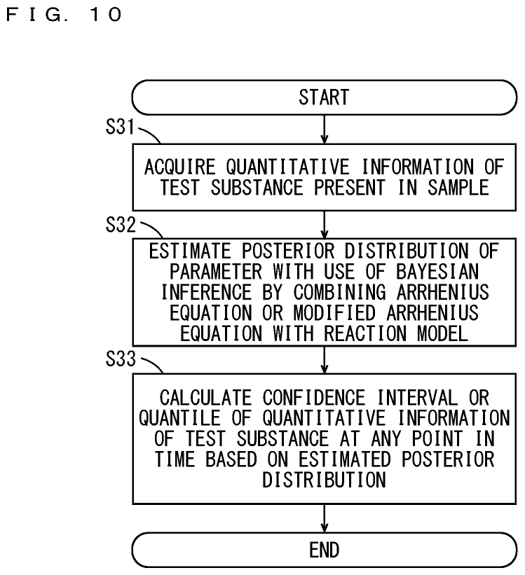

FIG. 9 is a flowchart according to the modified example 1. The steps S21 and S22 are similar to the steps S11 and S12 described with reference to FIG. 6. In the step S21, the acquirer 21 acquires data in regard to the ratio of a peak area of impurities with respect to a peak area of an active ingredient included in a pharmaceutical. In the step S12, the estimator 22 estimates a posterior distribution of a parameter of a generalized reaction model obtained by generalization of a plurality of reaction models with use of the Bayesian inference.

In the step S23, the calculator 23 calculates a confidence interval or a quantile in a period of time until quantitative information of a test substance reaches a predetermined specification limit based on the posterior distribution of a parameter estimated by the estimator 22. For example, the calculator 23 calculates a confidence interval or a quantile in the number of days (time) until a peak area ratio of impurities reaches a predetermined specification limit. Thus, in a case in which an allowable value of a peak area ratio of impurities of a pharmaceutical is defined, a confidence interval or a quantile of an effective shelf-life of a pharmaceutical can be presented.

### (6) Modified Example 2 of First Embodiment

With the analysis method using the Bayesian inference according to the first embodiment, more efficient estimation is possible by determination of an accelerative reaction. In a case in which the quadratic approximation of the measurement data MD is positive, a reaction model function has a characteristic of projecting downwardly. This indicates that the reaction of a sample proceeds in an accelerated manner. As such, the measurement data MD is subjected to the quadratic approximation, and a process is switched depending on whether it is possible to reject that a secondary coefficient is positive.

(6-1: Case in which it can be Rejected that Secondary Coefficient is Positive)

A reaction model representing an accelerative reaction is excluded from candidates for a reaction model, and the Bayesian inference is performed. Specifically, in case of generalization by summation, an accelerative reaction model is excluded from the candidates. Further, in case of generalization by inclusion, a range of parameters is restricted, or some parameters are deleted. Thus, in a case in which possibility of an accelerative reaction in the measurement data MD can be rejected, it is possible to perform the Bayesian inference with higher accuracy by limiting a generalized reaction model.

(6-2: Case in which it Cannot be Rejected that Secondary Coefficient is Positive but Active Component is Decomposed to Reference Value Under Certain Temperature and Humidity Conditions)

The Bayesian inference is performed with an accelerative reaction model included. This is because an active component has already been decomposed to a reference value, and thus a confidence interval of a posterior distribution is unlikely to be more greatly widened.

(6-3: Case in which it Cannot be Rejected that Secondary Coefficient is Positive but Active Component has not been Decomposed to Reference Value)

In a case in which the Bayesian inference is performed with an accelerative reaction model included, a confidence interval of a posterior distribution may be greatly widened. In this case, it is difficult to present a practical confidence interval or quantile. Therefore, acquisition of the measurement data MD is continued until decomposition of the active ingredient reaches the reference value. Determinations of (6-1) to (6-3) are carried out again while the acquisition of the measurement data MD is continued.

In this manner, with the modified example 2, it is possible to improve the accuracy of a result of estimation with use of the Bayesian inference by rejecting a case of an accelerative reaction. In a case in which extrapolation is performed along the time axis while an accelerative reaction model is used as it is, there is a problem that a subtle error becomes larger with an elapse of time and a confidence interval is greatly widened. However, such a problem can be avoided according to the modified example 2.

### (7) Modified Example 3 of First Embodiment

The modified example 3 is a method of stochastically selecting a plurality of reaction models for construction of a generalized reaction model.

(7-1: Two or More Reaction Models are Summed and Probability of Selection of Each Reaction Model is Acquired.)

As described above, a generalized reaction model is constructed by summation or inclusion. At this time, it is possible to obtain the probability in which an actual reaction is based on a reaction model by adding a new parameter or changing the setting of a distribution. For example, in a case in which the P2 model and the D1 model in a differential form are candidates, a discrete parameter p taking 2 values of (0, 1) is newly added as expressed by the formula 5.

$$[ Formula 5 ]$$

$$\frac{d α}{d t} = p {k}_{1} \cdot 2 {α}^{\frac{1}{2}} + ( 1 - p ) {k}_{2} \cdot \frac{1}{2 α} p ∈ \{ 0 , 1 \} \qquad ( 5 )$$

The Bayesian inference is performed on the generalized reaction model expressed by the formula 5, so that a distribution of p is obtained. However, p is a discrete parameter and takes 0 or 1. In a case in which the “probability of p=0” is the probability that the D1 model is selected and the “probability of p=1” is the probability that the P2 model is selected, the probability in which the measurement data MD is based on a particular model is obtained. The same applies to a case of generalization based on three or more reaction models. Further, a generalized reaction model expressed by the formula 6 can be constructed.

$$[ Formula 6 ]$$

$$\frac{d α}{d t} = {p}_{1} {k}_{1} \cdot 2 {α}^{\frac{1}{2}} + {p}_{2} {k}_{2} \cdot \frac{1}{2 α} ( {p}_{1} , {p}_{2} ) ∈ \{ ( 0,1 ) , ( 1,0 ) , ( 1,1 ) \} \qquad ( 6 )$$

With the formula 6, the “probability of p₁=0, p₂=1” and the “probability of p₁=1, p₂=0” and the “probability of p₁=1, p₂=1” are obtained. Thus, it is also possible to include a parallel pattern as a candidate. Further, instead of adding the discrete parameter p, a method of imposing a spike and slab distribution (a combination of a continuous distribution and a discrete probability of taking 0) on k may be considered.

(7-2: Order Parameter of Generalized Model is Discretized, and Probability of Selection of Each Model is Acquired.)

For example, in a case in which the P2 model and the D1 model in a differential form are candidates, the following generalized model with use of parameters (c, m, n) is considered, as expressed by the formula 7.

[Formula 7]

f(α)=cα^{m}(1−α)ⁿ (7)

At this time, the P2 model f(α)=2α\hat{ }(½) can be expressed as (c, m, n)=(2, 0.5, 0), and the D1 model f(α)=1/(2α) can be expressed as (c, m, n)=(0.5, −1, 0). Therefore, similarly to (7-1), it is possible to obtain the probability representing a particular reaction model by constructing a generalized reaction model as expressed by the formula 8 and discretizing a predictive value of the parameters (c, m, n) to perform the Bayesian inference.

$$[ Formula 8 ]$$

$$\frac{d α}{d t} = {{kc α}^{m} ( 1 - α )}^{n} ( c , m , n ) ∈ \{ ( 2 , 0.5 , 0 ) , ( 0 \text{.5} , - 1 , 0 ) \} \qquad ( 8 )$$

(7-3: Two or More Prior Distributions are Prepared and Probability of Taking Prior Distribution Set is Acquired.)

For example, in a case in which the P2 model and the D1 model in a differential form are candidates, a generalized model expressed by the formula 9 in which the parameters (c, m, n) are used is considered similarly to (7-1).

[Formula 9]

f(α)=cα^{m}(1−α)ⁿ (9)

At this time, it is possible to execute a similar process instead of discretizing predictive values of the parameters (c, m, n) by preparing a plurality of sets of prior distributions of the parameters (c, m, n). As an extreme example, a prior distribution X1 (other parameters are appropriately set) in which each of (c, m, n) is distributed to only one point of (2, 0.5, 0) and a prior distribution X2 (other parameters are set similar to the prior distribution X1) in which each of (c, m, n) is distributed to only one point of (0.5, −1, 0) are prepared, and estimation is carried out on the assumption that one of the two prior distributions is to be selected, the probability that two prior distribution sets are taken can be acquired. Thus, an effect similar to that of (7-2) is obtained. Further, in a case in which a prior distribution is set, it is possible to designate not only one point but also a distribution. Therefore, it is possible to designate a model function “group” having a wide range, and it is possible to set an appropriate prior distribution with respect to parameters other than the parameters (c, m, n) for each group. Further, such a generalized formula of summation described in (7-1) can be used similarly for a prior distribution.

### (8) Second Embodiment

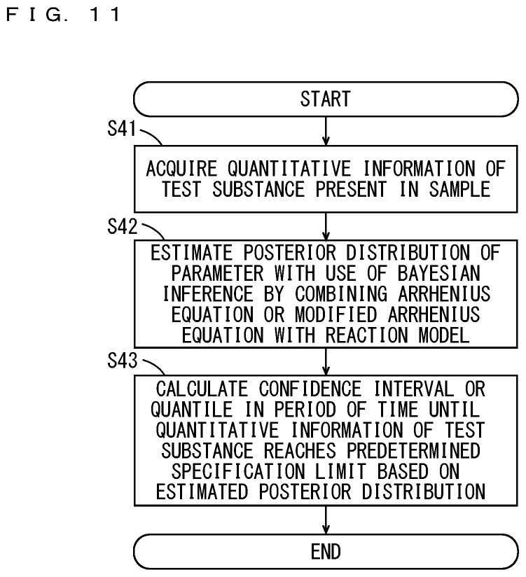

Next, an analysis method according to a second embodiment will be described with reference to the flow chart of FIG. 10. The analysis method of the second embodiment is extrapolation along the temperature axis with use of the Arrhenius equation or the modified Arrhenius equation described in (3-2). The flowchart of FIG. 10 shows a process executed by the CPU 11 shown in FIG. 1.

In the step S31, the acquirer 21 acquires quantitative information of a test substance present in a sample. Specifically, the acquirer 21 acquires data in regard to the ratio of a peak area of impurities with respect to a peak area of an active ingredient included in a pharmaceutical. Here, the measurement date MD acquired in step S31 is the data acquired under high temperature conditions (severe conditions). Next, in the step S32, the estimator 22 applies the Arrhenius equation (formula 1) or the modified Arrhenius equation (formula 2) to the reaction model formula (Formula 8), and estimates a posterior distribution of parameters (A, E, B, etc.) related to the temperature axis and the humidity axis and parameters (m, n, etc.) for determining a reaction model with use of the Bayesian inference. Thus, the measurement data MD is extrapolated along the temperature axis and is extrapolated along the time axis direction, and a confidence interval or a quantile of a peak area ratio at any point in time under a low temperature condition (normal storage condition) can be calculated. In this manner, the estimator 22 estimates a posterior distribution of a parameter with use of the Bayesian inference by combining the Arrhenius equation or the modified Arrhenius equation with the reaction model. The Arrhenius equation, the modified Arrhenius equation and a plurality of reaction models are stored in the storage device 16.

Next, in the step S33, the calculator 23 calculates a confidence interval or a quantile of quantitative information of a test substance at any point in time based on the posterior distribution of a parameter estimated by the estimator 22. That is, the calculator 23 calculates a confidence interval or a quantile of a value of peak area ratio at any point in time. For example, the calculator 23 calculates a confidence interval or a quantile at a point in time such as one year later, two years later or three years later in regard to a peak area ratio of impurities with respect to an active ingredient included in a pharmaceutical. The calculated confidence interval or the calculated quantile may be displayed in the display 15 by the outputter 24.

### (9) Modified Example of Second Embodiment

In the second embodiment, the calculator 23 calculates a confidence interval or a quantile of quantitative information of a test substance at any point in time based on the posterior distribution of a parameter estimated by the estimator 22. In the modified example of the second embodiment, a confidence interval or the like in a period of time until quantitative information of a test substance reaches a predetermined specification limit is calculated.

FIG. 11 is a flowchart according to the modified example. The steps S41 and S42 are similar to the steps S31 and S32 described with reference to FIG. 10. In the step S43, the calculator 23 calculates a confidence interval or a quantile in a period of time until the quantitative information of a test substance reaches a predetermined specification limit based on a posterior distribution of a parameter estimated by the estimator 22. For example, the calculator 23 calculates a confidence interval or a quantile in the number of days (time) until a peak area ratio of impurities reaches a predetermined specification limit. Thus, in a case in which an allowable value of a peak area ratio of impurities of a pharmaceutical is defined, a confidence interval or a quantile of an effective shelf-life of a pharmaceutical can be presented.

### (10) Other Modified Examples

In each above-mentioned embodiment, the sample analysis device 1 is a pharmaceutical analysis device, by way of example. The sample analysis device 1 of the present embodiment can be utilized to acquire quantitative information of a test substance in various samples other than pharmaceuticals. The list of reaction models shown in FIG. 4 is one example. The reaction model to which the analysis method in the present embodiment is applied is limited in particular.

### (11) Aspects

It is understood by those skilled in the art that the plurality of above-mentioned illustrative embodiments are specific examples of the below-mentioned aspects.

(Item 1) A sample analysis device according to one aspect includes an acquirer that acquires quantitative information of a test substance present in a sample, an estimator that reads a generalized reaction model obtained by generalization of a plurality of reaction models from a storage device and estimates a posterior distribution of a parameter of the generalized reaction model using Bayesian inference, and a calculator that calculates a confidence interval or a quantile of the quantitative information of a test substance in any period of time or calculates a confidence interval of a quantile in a period of time until the quantitative information of a test substance reaches a predetermined specification limit, based on the posterior distribution of a parameter estimated by the estimator.

The reliability of a result of estimation with use of the Bayesian inference can be improved.

(Item 2) The sample analysis device according to item 1, wherein the estimator may stochastically select the plurality of reaction models.

The reliability of a result of estimation with use of the Bayesian inference can be improved.

(Item 3) The sample analysis device according to item 1, wherein the estimator may estimate a posterior distribution using the Bayesian inference and may select the plurality of reaction models based on the estimated posterior distribution by setting a combination of the plurality of reaction models as a plurality of prior distributions.

The reliability of a result of estimation with use of the Bayesian inference can be improved.

(Item 4) The sample analysis device according to any one of items 1 to 3, wherein the generalized reaction model may be obtained by summation of the plurality of reaction models.

An appropriate reaction model can also be applied to a complex reaction.

(Item 5) The sample analysis device according to any one of items 1 to 3, wherein the generalized reaction model may be one model that includes the plurality of reaction models.

An appropriate reaction model can also be applied to a complex reaction.

(Item 6) The sample analysis device according to any one of items 1 to 5, wherein the estimator may switch the plurality of reaction models to be applied in accordance with whether reaction of the sample includes accelerated reaction.

The accuracy of a result of estimation with use of the Bayesian inference can be improved.

(Item 7) The sample analysis device according to item 6, wherein the estimator may use quadratic approximation to determine whether accelerated reaction is included.

The accuracy of a result of estimation with use of the Bayesian inference can be improved.

(Item 8) A sample analysis device according to another aspect includes an acquirer that acquires quantitative information of a test substance present in a sample, an estimator that reads a reaction model stored in a storage device and estimates a posterior distribution of a parameter using Bayesian inference by combining an Arrhenius equation or a modified Arrhenius equation with the reaction model, and a calculator that calculates a confidence interval or a quantile of the quantitative information of a test substance in any period of time or calculates a confidence interval of a quantile in a period of time until the quantitative information of a test substance reaches a predetermined specification limit, based on the posterior distribution of a parameter estimated by the estimator.

A reasonable confidence interval can be presented while a period of time required for acquisition of necessary data is shortened.

(Item 9) A sample analysis method according to another aspect includes acquiring quantitative information of a test substance present in a sample, reading a generalized reaction model obtained by generalization of a plurality of reaction models from a storage device and estimating a posterior distribution of a parameter of the generalized reaction model using Bayesian inference, and calculating a confidence interval or a quantile of the quantitative information of a test substance in any period of time or calculating a confidence interval of a quantile in a period of time until the quantitative information of a test substance reaches a predetermined specification limit, based on the estimated posterior distribution of a parameter.

The reliability of a result of estimation with use of the Bayesian inference can be improved.

(Item 10) A sample analysis method according to another aspect includes acquiring quantitative information of a test substance present in a sample, reading a reaction model stored in a storage device and estimating a posterior distribution of a parameter using Bayesian inference by combining an Arrhenius equation or a modified Arrhenius equation with the reaction model, and calculating a confidence interval or a quantile of the quantitative information of a test substance in any period of time or calculating a confidence interval of a quantile in a period of time until the quantitative information of a test substance reaches a predetermined specification limit, based on the estimated posterior distribution of a parameter.

A reasonable confidence interval can be presented while a period of time required for acquisition of necessary data is shortened.

(Item 11) A pharmaceutical analysis device according to another aspect, wherein the sample includes a formulation or a drug substance, and the test substance includes an active ingredient or impurities present in the formulation or the drug substance, in the sample analysis device according to item 1.

The reliability of a result of estimation with use of the Bayesian inference can be improved.

(Item 12) The pharmaceutical analysis device according to another aspect, wherein the sample includes a formulation or a drug substance, and the test substance includes an active ingredient or impurities present in the formulation or the drug substance, in the sample analysis device according to item 8.

The reliability of a result of estimation with use of the Bayesian inference can be improved.

(Item 13) A pharmaceutical analysis method according to another aspect, wherein the sample includes a formulation or a drug substance, and the test substance includes an active ingredient or impurities present in the formulation or the drug substance, in the sample analysis method according to item 9.

The reliability of a result of estimation with use of the Bayesian inference can be improved.

(Item 14) A pharmaceutical analysis method according to another aspect, wherein the sample includes a formulation or a drug substance, and the test substance includes an active ingredient or impurities present in the formulation or the drug substance, in the sample analysis method according to item 10.

The reliability of a result of estimation with use of the Bayesian inference can be improved.

While preferred embodiments of the present disclosure have been described above, it is to be understood that variations and modifications will be apparent to those skilled in the art without departing the scope and spirit of the present disclosure. The scope of the present disclosure, therefore, is to be determined solely by the following claims.

I/We claim:

1. A sample analysis device comprising:
an acquirer that acquires quantitative information of a test substance present in a sample;
an estimator that reads a generalized reaction model obtained by generalization of a plurality of reaction models from a storage device and estimates a posterior distribution of a parameter of the generalized reaction model using Bayesian inference; and
a calculator that calculates a confidence interval or a quantile of the quantitative information of a test substance in any period of time or calculates a confidence interval of a quantile in a period of time until the quantitative information of a test substance reaches a predetermined specification limit, based on the posterior distribution of a parameter estimated by the estimator.

2. The sample analysis device according to claim 1, wherein
the estimator stochastically selects the plurality of reaction models.

3. The sample analysis device according to claim 1, wherein
the estimator estimates a posterior distribution using the Bayesian inference and selects the plurality of reaction models based on the estimated posterior distribution by setting a combination of the plurality of reaction models as a plurality of prior distributions.

4. The sample analysis device according to claim 1, wherein
the generalized reaction model is obtained by summation of the plurality of reaction models.

5. The sample analysis device according to claim 1, wherein
the generalized reaction model is one model that includes the plurality of reaction models.

6. The sample analysis device according to claim 1, wherein
the estimator switches the plurality of reaction models to be applied in accordance with whether reaction of the sample includes accelerated reaction.

7. The sample analysis device according to claim 6, wherein
the estimator uses quadratic approximation to determine whether accelerated reaction is included.

8. A sample analysis device comprising:
an acquirer that acquires quantitative information of a test substance present in a sample;
an estimator that reads a reaction model stored in a storage device and estimates a posterior distribution of a parameter using Bayesian inference by combining an Arrhenius equation or a modified Arrhenius equation with the reaction model; and
a calculator that calculates a confidence interval or a quantile of the quantitative information of a test substance in any period of time or calculates a confidence interval of a quantile in a period of time until the quantitative information of a test substance reaches a predetermined specification limit, based on the posterior distribution of a parameter estimated by the estimator.

9. A sample analysis method including:
acquiring quantitative information of a test substance present in a sample;
reading a generalized reaction model obtained by generalization of a plurality of reaction models from a storage device and estimating a posterior distribution of a parameter of the generalized reaction model using Bayesian inference; and
calculating a confidence interval or a quantile of the quantitative information of a test substance in any period of time or calculating a confidence interval of a quantile in a period of time until the quantitative information of a test substance reaches a predetermined specification limit, based on the estimated posterior distribution of a parameter.

10. A sample analysis method including:
acquiring quantitative information of a test substance present in a sample;
reading a reaction model stored in a storage device and estimating a posterior distribution of a parameter using Bayesian inference by combining an Arrhenius equation or a modified Arrhenius equation with the reaction model; and
calculating a confidence interval or a quantile of the quantitative information of a test substance in any period of time or calculating a confidence interval of a quantile in a period of time until the quantitative information of a test substance reaches a predetermined specification limit, based on the estimated posterior distribution of a parameter.

11. A pharmaceutical analysis device, wherein
the sample includes a formulation or a drug substance, and the test substance includes an active ingredient or impurities present in the formulation or the drug substance, in the sample analysis device according to claim 1.

12. The pharmaceutical analysis device, wherein
the sample includes a formulation or a drug substance, and the test substance includes an active ingredient or impurities present in the formulation or the drug substance, in the sample analysis device according to claim 8.

13. A pharmaceutical analysis method, wherein
the sample includes a formulation or a drug substance, and the test substance includes an active ingredient or impurities present in the formulation or the drug substance, in the sample analysis method according to claim 9.

14. A pharmaceutical analysis method, wherein
the sample includes a formulation or a drug substance, and the test substance includes an active ingredient or impurities present in the formulation or the drug substance, in the sample analysis method according to claim 10.
